(19) United States
(12) Patent Application Publication
(10) Pub. No.: US 2006/0173430 A1
(43) Pub. Date: Aug. 3, 2006

(54) Fluid-absorbing products comprising superabsorbent material in a water-soluble pouch and methods of using the same

(76) Inventors: Baosheng Lee, Duluth, GA (US); Youzhen Ding, Norcross, GA (US); Barbara Osborne, Alpharetta, GA (US); Debbie Wooley, Birmingham, AL (US); Lindy Radaszewski, Brookfield, IL (US)

Correspondence Address: WITHERS & KEYS, LLC, P. O. BOX 2049, MCDONOUGH, GA, 30253, US

(21) Appl. No.: 11/050,510

(22) Filed: Feb. 3, 2005

(51) Int. Cl. A61F 13/15

(52) U.S. Cl. 604/368; 604/360; 604/364

(57) ABSTRACT

Fluid-absorbing articles containing superabsorbent material in a water-soluble pouch are disclosed. Methods of making and using fluid-absorbing articles are also disclosed.

### FIELD OF THE INVENTION

The present invention relates to fluid-absorbing articles comprising superabsorbent material in a water-soluble pouch. The present invention further relates to methods of using fluid-absorbing articles comprising superabsorbent material in a water-soluble pouch.

### BACKGROUND OF THE INVENTION

There exists a need in the art for fluid-absorbing articles capable of immobilizing aqueous solutions and components within the aqueous solutions.

### SUMMARY OF THE INVENTION

The present invention addresses some of the needs in the art by the discovery of fluid-absorbing products, which may be used to immobilizing an aqueous solution and components within the aqueous solution. In one exemplary embodiment of the present invention, the fluid-absorbing article comprises (i) particulate superabsorbent material; and (ii) a sealed water-soluble pouch encapsulating the particulate superabsorbent material, wherein the sealed water-soluble pouch is formed around the particulate superabsorbent material to minimize the amount of air within the sealed water-soluble pouch.

In a further exemplary embodiment of the present invention, the fluid-absorbing article consists essentially of (i) particulate superabsorbent material, and (ii) a water-soluble pouch encapsulating the particulate superabsorbent material, wherein the water-soluble pouch consists essentially of a water-soluble film.

In yet a further exemplary embodiment of the present invention, the fluid-absorbing article consisting essentially of a mixture of (a) particulate superabsorbent material and (b) at least one of (i) a particulate antimicrobial component, (ii) an antimicrobial component coated onto the particulate superabsorbent material, and (iii) an antimicrobial component coated onto a particulate support consisting of carbon black; and a water-soluble pouch encapsulating the mixture, wherein the water-soluble pouch consists essentially of water-soluble film material.

The present invention is further directed to methods of making fluid-absorbing articles. In one exemplary embodiment of the present invention, the method of making a fluid-absorbing article comprises the steps of (i) surrounding a mass of particulate superabsorbent material with at least one sheet of water-soluble film; and (ii) forming a seal around the mass to encapsulate the particulate superabsorbent material in a sealed pouch having a pouch volume. The method may further comprise one or more additional steps including, but not limited to, removing any air trapped within water-soluble film prior to forming the seal, heating the water-soluble film and mass of particulate superabsorbent material, applying a liquid material onto a portion of the water-soluble film to enhance the sealing step, and incorporating at least one antimicrobial component into the sealed pouch.

The present invention is even further directed to methods of using fluid-absorbing articles to immobilize fluids. In one exemplary embodiment of the present invention, the method of using a fluid-absorbing article comprises the steps of (i) introducing a fluid-absorbing article into an aqueous solution, wherein the fluid-absorbing article consists essentially of (a) particulate superabsorbent material, and (b) a water-soluble pouch encapsulating the particulate superabsorbent material, wherein the water-soluble pouch consists essentially of water-soluble film.

In a further exemplary embodiment of the present invention, the method of using a fluid-absorbing article comprises the steps of (i) collecting one or more fluids in a container; and (ii) introducing a fluid-absorbing article into the container, wherein the fluid-absorbing article comprises (a) particulate superabsorbent material; and (b) a sealed water-soluble pouch encapsulating the particulate superabsorbent material, wherein the sealed water-soluble pouch is formed around the particulate superabsorbent material to minimize the amount of air within the sealed water-soluble pouch.

These and other features and advantages of the present invention will become apparent after a review of the following detailed description of the disclosed embodiments and the appended claims.

### BRIEF DESCRIPTION OF THE FIGURES

FIG. 1 depicts an exemplary fluid-absorbing article of the present invention; and

FIG. 2 depicts a cross-sectional view of the exemplary fluid-absorbing article of FIG. 1 along line A-A shown in FIG. 1.

### DETAILED DESCRIPTION OF THE INVENTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to fluid-absorbing articles capable of immobilizing an aqueous solution and components therein. The present invention is further directed to methods of making fluid-absorbing articles, as well as methods of using the fluid-absorbing articles in a variety of applications including, but not limited to, medical applications. A description of the various embodiments of the present invention is provided below.

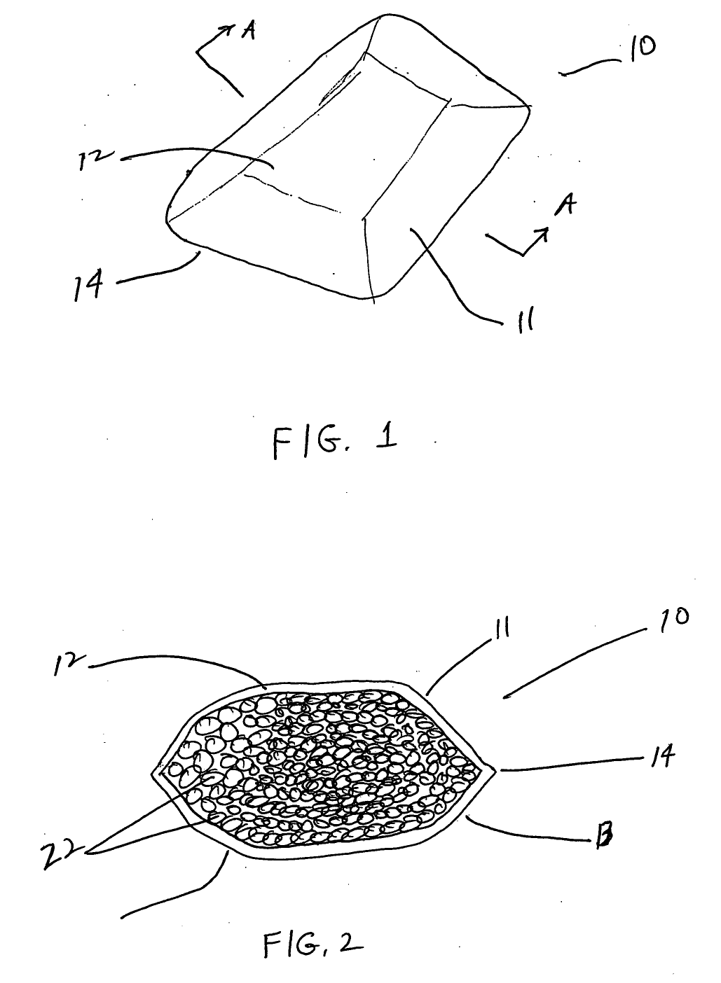

An exemplary fluid-absorbing article of the present invention is shown in FIGS. 1-2. As shown in FIG. 1, exemplary fluid-absorbing article 10 comprises a pouch 11 having an upper surface 12, a lower surface 13 (see FIG. 2), and an outer periphery 14. FIG. 2 depicts a cross-sectional view of exemplary fluid-absorbing article 10 along line A-A shown in FIG. 1.

As shown in FIG. 2, exemplary fluid-absorbing article 10 comprises pouch 11 having upper surface 12, lower surface 13, and outer periphery 14, as well as superabsorbent particles 22 contained within pouch 11. As discussed below, pouch 11 may include other materials such as an antimicrobial component.

I. Water-Soluble Pouch Products

The present invention is directed to fluid-absorbing articles such as exemplary fluid-absorbing article 10 shown in FIGS. 1-2. The fluid-absorbing articles may comprise one or more components as described below.

A. Water-Soluble Pouch Material

The fluid-absorbing articles of the present invention comprise a sealable pouch formed from at least one water-soluble film. As used herein, the term “water-soluble” refers to materials having a degree of solubility in water at a water temperature of about 20° C. (68° F.) or above. Upon exposure to water, the water-soluble film material solubilizes within a desired time period, typically within seconds. Desirably, upon exposure to water, at least a portion of the water-soluble film material solubilizes within about 10 seconds (about 8 seconds, about 6 seconds, about 5 seconds, about 4 seconds, about 3 seconds, about 2 seconds, or about 1 second) such that the pouch contents (e.g., the superabsorbent particles and any other encapsulated materials) are exposed to the water within about 10 seconds (about 8 seconds, about 6 seconds, about 5 seconds, about 4 seconds, about 3 seconds, about 2 seconds, or about 1 second).

Suitable water-soluble film-forming materials for use in the present invention include, but are not limited to, polyvinyl alcohol; polyacrylic acid; polymethacrylic acid; polyacrylamide; water-soluble cellulose derivatives such as methyl celluloses, ethyl celluloses, hydroxymethyl celluloses, hydroxypropyl methyl celluloses, and carboxymethyl celluloses; carboxymethylchitin; polyvinyl pyrrolidone; ester gum; water-soluble derivatives of starch such as hydroxypropyl starch and carboxymethyl starch; and water-soluble polyethylene oxides.

In one desired embodiment, the water-soluble film material comprises polyvinyl alcohol with or without acetyl groups, cross-linked or uncross-linked. Suitable polyvinyl alcohol materials are described in U.S. Pat. Nos. 5,181,967; 5,207,837; 5,268,222; 5,620,786; 5,885,907; 5,891,812; the disclosures of all of which are hereby incorporated in their entirety by reference.

The water-soluble film material has an average film thickness that may vary depending on a given application. Typically, the water-soluble film material has an average film thickness of up to about 100 microns (μm) (3.9 mil). In one exemplary embodiment of the present invention, the water-soluble film material has an average film thickness ranging from about 25 μm (1.0 mil) to about 76 μm (3.0 mil). In one desired embodiment of the present invention, the water-soluble film material has an average film thickness of about 38 μm (1.5 mil).

Suitable water-soluble film materials are commercially available from a number of sources. Suitable water-soluble film materials include, but are not limited to, PVA film commercially available under the trade designation MONOSOL® from Monosol USA (Portage, Ind.).

In one desired embodiment of the present invention, the water-soluble film material is “shrink-wrapped” to encapsulate one or more materials within a sealed pouch formed from the water-soluble film material. In this embodiment, one or more sheets of water-soluble film material are (i) positioned to surround one or more materials (described below), and (ii) subsequently sealed to form a pouch having a minimal amount of air and/or empty space within the sealed pouch. Various process parameters may be used to minimize the amount of air and/or empty space within the pouch. In one exemplary embodiment, heat is used to shrink the water-soluble film material so that the water-soluble film material substantially conforms to the shape of the contents within the sealed pouch. In a further exemplary embodiment, a vacuum may be used to remove excess air from the pouch so that the water-soluble film material substantially conforms to the shape of the contents within the sealed pouch. Once the excess air is removed from the pouch, the pouch is sealed using any known sealing technique.

In this embodiment of the present invention, the shrink-wrapped fluid-absorbing product comprises (or consists essentially of or consists of) water-soluble film material, and one or more materials sealed within the water-soluble film material to form a pouch having a minimal amount of air and/or empty space within the sealed pouch. Desirably, the amount of air within the sealed pouch is less than about 10% of the pouch volume, more desirably, less than about 8.0% (about 7.0%, about 6.0%, about 5.0%, about 4.0%, about 3.0%, about 2.0%, about 1.0%, about 0.5%) of the pouch volume, based on a total pouch volume.

In some embodiments of the present invention, the water-soluble film comprises water-soluble material alone or in combination with water-insoluble material (e.g., inorganic fillers, pigments, etc.). When water-insoluble materials are used to form the water-soluble film, desirably less than about 50 parts by weight (pbw) of water-insoluble material is used in combination with at least about 50 parts by weight (pbw) of one or more of the above-mentioned water-soluble materials to form the water-soluble film, based on a total parts by weight of the water-soluble film. More desirably, the water-soluble film comprises at least about 70 pbw of water-soluble material and less than about 30 pbw of water-insoluble material, even more desirably, at least about 90 pbw of water-soluble material and less than about 10 pbw of water-insoluble material, and even more desirably, at least about 98 pbw of water-soluble material and less than about 2 pbw of water-insoluble material, based on a total parts by weight of the water-soluble film.

In a further embodiment, the water-soluble film used to form the fluid-absorbing articles of the present invention consists essentially of water-soluble material. In yet a further embodiment, the water-soluble film used to form the fluid-absorbing articles of the present invention consists of water-soluble material.

B. Superabsorbent Material

The fluid-absorbing articles of the present invention further comprise superabsorbent material. The superabsorbent material comprises polymeric or polymerizable material that swells upon exposure to water to form a hydrated gel (i.e., a hydrogel) by absorbing large amounts of water. As used herein, the term “superabsorbent” is used to describe materials that absorb large quantities of liquid, typically, in excess of 10 to 15 parts of liquid per part of superabsorbent material. Superabsorbent materials generally fall into three classes: (1) starch graft copolymers, (2) cross-linked carboxymethylcellulose derivatives, and (3) modified hydrophilic polyacrylates. Examples of such superabsorbent materials include, but are not limited to, hydrolyzed starch-acrylonitrile graft copolymer, a neutralized starch-acrylic acid graft copolymer, a saponified acrylic acid ester-vinyl acetate copolymer, a hydrolyzed acrylonitrile copolymer, a hydrolyzed acrylamide copolymer, a modified cross-linked polyvinyl alcohol, a neutralized self-crosslinking polyacrylic acid, a cross-linked polyacrylate salt, carboxylated cellulose, and a neutralized cross-linked isobutylenemaleic anhydride copolymer. Desirably, the superabsorbent material comprises sodium polyacrylate.

Superabsorbent material is commercially available in a variety of forms including, but not limited to, particulate material, and fibrous material. In the present invention, the superabsorbent material is desirably in the form of particulate material. The superabsorbent particles may have an average particle size that varies depending on a given application. Typically, the superabsorbent particles have an average particle size ranging from about 10 microns (μm) to about 10,000 μm, desirably, ranging from about 100 μm to about 1000 μm, even more desirably, from about 150 μm to about 800 μm.

Superabsorbent particles are commercially available from a number of sources including, but not limited to, starch graft polyacrylate hydrogel fine particles commercially available under the trade designation SANWET from Hoechst-Celanese (Portsmouth, Va.); superabsorbent particles commercially available under the trade designation FAVOR from Stockhausen (Greensboro, N.C.); superabsorbent particles commercially available under the trade designations NORSOCRYL and LIQUIBLOCK from Emerging Technologies, Inc. (Greensboro, N.C.); and superabsorbent particles commercially available from BASF (Mount Olive, N.J.).

In one desired embodiment of the present invention, the fluid-absorbing article consists essentially of (i) particulate superabsorbent material, and (ii) water-soluble film material encapsulating the particulate superabsorbent material, wherein the water-soluble film material forms a sealed pouch having a pouch volume. In this embodiment, the fluid-absorbing article does not contain any other components other than possible impurities and a minimal amount of air and water. Desirably, the fluid-absorbing article contains less than about 5.0 wt % water (less than about 4.0, about 3.0, about 2.0, about 1.0, about 0.5, about 0.1 wt % water) based on a total weight of the fluid-absorbing article.

C. Antimicrobial Agents

The fluid-absorbing article may further comprise one or more antimicrobial components. The antimicrobial component provides germicidal action that kills microorganisms within a given aqueous solution. Any known solid antimicrobial material may be used in the present invention. Suitable antimicrobial components include, but are not limited to, dialdehydes; 2,2-dibromo-3-nitrilopropionamide (DBNPA) commercially available from Dow Chemical (Midland, Mich.); a concentrate of 10,10′-oxybisphenoxarsine (OBPA) commercially available from Rohm & Haas (Philadelphia, Pa.) under the trade designation VINYZENE SB-1 PS; or a combination thereof.

In one exemplary embodiment of the present invention, the antimicrobial component used in the present invention comprises a dialdehyde having a chemical formula, OHC—R—CHO, wherein R comprises a C₁ to C₆ alkylene group; a substituted C₁ to C₆ alkylene group substituted with moieties such as benzyl groups; or a single covalent bond. The R group may be selected from moieties including, but not limited to, lower alkylenes of methylene, ethylene, propylene and butylenes, or can be a single covalent bond between the two outer carbonyl groups. Examples of suitable compounds having the above formula include, but not limited to, succinaldehyde, malonaldehyde, adipaldehyde, glyoxal, glutaraldehyde, and orthophthaldehyde.

When used as the antimicrobial component, the one or more dialdehydes may be coated onto a particulate substrate desirably having a particle size similar to the above-mentioned particle size of the particulate superabsorbent material, and blended into a mixture containing the particulate superabsorbent material. Suitable substrates include, but are not limited to, carbon black. Alternatively, the one or more dialdehydes may be coated directly onto the particulate superabsorbent material.

When present, the antimicrobial component or components are typically present in an amount of up to about 20.0 wt % based on a total weight of the encapsulated material (i.e., the superabsorbent material and any additional components). In one exemplary embodiment, the antimicrobial component or components are present in an amount ranging from about 5.0 wt % to about 15.0 wt %, more desirably, from about 8.0 wt % to about 12.0 wt %, and even more desirably, about 10.2 wt % based on a total weight of the encapsulated material.

In one exemplary embodiment of the present invention, glutaraldehyde is used alone or in combination with orthophthaldehyde as the antimicrobial component or components. In this embodiment, glutaraldehyde is typically present in an amount of up to about 20.0 wt %, desirably, from about 8.0 wt % to about 12.0 wt %, while orthophthaldehyde is typically present in an amount of up to about 10.0 wt %, desirably, from about 1.0 wt % to about 8.0 wt % based on a total weight of the encapsulated material. The glutaraldehyde and/or orthophthaldehyde may be coated onto a carbon black substrate or directly onto the particulate superabsorbent particles.

Antimicrobial dialdehydes are commercially available from a number of sources. Suitable antimicrobial dialdehydes for use in the present invention include, but are not limited to, antimicrobial dialdehydes commercially available under the trade designation UCARCIDE™ from Dow Chemical (Midland, Mich.), such as UCARCIDE™ 250 and UCARCIDE™ 750.

As discussed above, the fluid-absorbing article may comprise (or consist essentially of or consist of) water-soluble film material, and one or more of the above-mentioned encapsulated materials sealed within the water-soluble film material to form a pouch having a minimal amount of air and/or water within the sealed pouch. Regardless of the encapsulated materials in this exemplary embodiment, the amount of air within the sealed pouch is desirably less than about 10% of the pouch volume, more desirably, less than about 8.0% (about 7.0%, about 6.0%, about 5.0%, about 4.0%, about 3.0%, about 2.0%, about 1.0%, about 0.5%) of the pouch volume, based on a total pouch volume.

II. Methods of Making Water-Soluble SAP Pouch Products

The present invention is further directed to methods of making fluid-absorbing articles. In one exemplary embodiment of the present invention, the method of making a fluid-absorbing article comprises the steps of (i) providing one or more sheets of water-soluble film material; (ii) placing particulate superabsorbent material onto a first sheet of water-soluble film material; (i) placing a second sheet of water-soluble film material over the particulate superabsorbent material or (ii) folding a portion of the first sheet of water-soluble film material over the particulate superabsorbent material; and bonding (i) portions of the first sheet to the second sheet or (ii) portions of the first sheet to itself to form a water-soluble pouch containing the particulate superabsorbent material.

The step of forming the water-soluble pouch may involve one or more of the following additional steps: cutting one or more pouch-forming sheets of water-soluble film material from a larger sheet of water-soluble film material; placing a sheet of water-soluble film material over a cavity; feeding particulate superabsorbent material into the water-soluble sheet-lined cavity; mixing one or more components with the particulate superabsorbent material; attaching a vacuum line to the cavity to remove air from the cavity; applying a bonding agent to a portion of the sheet of water-soluble film material to be bonded to another portion of a sheet of water-soluble film material; applying heat and/or pressure to portions of a sheet of water-soluble film material to be bonded; and applying heat to the water-soluble film material to shrink the water-soluble film material.

The step of bonding portions of adjacent pouch-forming sheets to one another may be performed using any bonding technique including, but not limited to, adhesive bonding, and thermal bonding. In one exemplary embodiment, water is used as a bonding agent, wherein water is sprayed onto portions of the pouch-forming sheets. After wetting outer surfaces of the pouch-forming sheets, pressure and/or heat may be applied to the pouch-forming sheets to bond sheet portions to one another.

In one exemplary embodiment of the present invention, the method of making fluid-absorbing articles further comprises one or more steps in order to minimize the amount of air and/or empty space within the water-soluble pouch. As discussed above, a vacuum may be used to remove excess air within a water-soluble pouch prior to sealing the pouch. Alternatively, a sealed water-soluble pouch may be punctured by a vacuum line, which removes air from the pouch. Once air is removed, the punctured water-soluble pouch may be resealed using any of the bonding methods described above. In a further exemplary embodiment of the present invention, heat may be used to shrink the water-soluble film around a mass of superabsorbent material and other optional encapsulated materials.

Any other known method may be used to form fluid-absorbing articles of the present invention having a minimum amount of air within the water-soluble pouch. For example, by choosing the appropriate (i) cavity size, (ii) amount of particulate superabsorbent material, and (iii) size of water-soluble film material, a fluid-absorbing article having a minimum amount of air within the water-soluble pouch may be formed without the need for a vacuum line and a heat-shrinking step.

For example, as described above, a first sheet of water-soluble film may be used to line a cavity having a shape of a finished pouch. Particulate superabsorbent material and other components may be fed into the cavity to occupy the entire volume of the cavity. A second sheet of water-soluble film may then be applied over the cavity and bonded to the first sheet of water-soluble film along edges of the cavity. The bonding step may take place moving from one side of the cavity to an opposite side of the cavity to allow air, if present, to be forced out of the cavity during and prior to a final bonding phase.

III. Methods of Using Water-Soluble SAP Pouch Products

The present invention is also directed to methods of using fluid-absorbing articles to immobilize fluids. In one exemplary embodiment of the present invention, the method of using a fluid-absorbing article comprises the steps of (i) introducing a fluid-absorbing article into an aqueous solution, wherein the fluid-absorbing article consists essentially of (a) particulate superabsorbent material, and (b) a water-soluble pouch encapsulating the particulate superabsorbent material, wherein the water-soluble pouch consists essentially of water-soluble film. Desirably, the water-soluble film is a PVA film.

In a further exemplary embodiment of the present invention, the method of using a fluid-absorbing article comprises the steps of (i) introducing a fluid-absorbing article into an aqueous solution, wherein the fluid-absorbing article comprises (a) particulate superabsorbent material, and (b) a water-soluble, shrink-wrapped film encapsulating the particulate superabsorbent material (i.e., a water-soluble film encapsulating the particulate superabsorbent material so as to minimize the amount of air or open volume within a sealed pouch formed by the water-soluble film). Desirably, the water-soluble, shrink-wrapped film is a PVA film.

In the methods of using a fluid-absorbing article to immobilize a given fluid, the water-soluble pouch may also contain additional components to further treat the given fluid. For example, at least one antimicrobial component may be added to the water-soluble pouch in order to kill microbes present in the given fluid and/or prevent the growth of microbes in the given fluid.

The fluid-absorbing articles may be used to immobilize a given fluid in any type of container including, but not limited to, a canister for use in collecting fluids, such as used in an operating room environment, a bag, or any other container capable of at least temporarily containing a fluid.

In one exemplary embodiment of the present invention, the fluid-absorbing articles are used to immobilize fluid contained in a canister, such as a canister for use in collecting fluids in an operating room environment. In this embodiment, the canister may contain one or more fluids generated during a procedure in an operating room including, but not limited to, body fluids (e.g., blood, urine, etc.), medicinal fluids, irrigation fluids, etc. Typically, a negative pressure is produced within the canister via a vacuum system, so as to pull one or more fluids into the canister. Once inside the canister, the fluids remain until disposal. In order to prevent potential contamination of the operating room during disposal of the fluid-containing canister, the fluid is immobilized by introducing a fluid-absorbing article of the present invention into the canister.

Canisters typically have a cylindrical configuration having a volume capacity of 1000 cubic centimeters (cc), 1500 cc, 3000 cc, etc., but may have any shape and/or size. The canister may have an inlet having a circular opening having a diameter of about 5.1 cm (2.0 in.). In one exemplary embodiment, a water-soluble pouch having a tubular or cylindrical shape and an outer diameter of up to about 5.0 cm (1.97 in.) may be introduced into the fluid-containing canister.

In further exemplary embodiments, the fluid-absorbing articles are used in urine containment systems and emesis collection systems. In such systems, urine and/or emesis may be collected in a container, such as a bag. Prior to or after introduction of urine and/or emesis into the container, a fluid-absorbing article of the present invention may be inputted into the container to immobilize the urine and/or emesis. Immobilization of the urine and/or emesis is advantageous prior to transporting the urine and/or emesis from a source to a disposal location.

In another exemplary embodiment, the fluid-absorbing articles described herein may be used in wound therapy procedures, such as negative pressure wound therapy (NPWT) procedures, wherein interstitial fluids and infectious materials are removed from a wound site and collected.

In yet a further exemplary embodiment, the fluid-absorbing articles described herein may be used in an emulsification receptor procedure, wherein a tumor is emulsified, and the emulsified solution is removed from a patient's body. The emulsified solution may be collected and then immobilized in a container, such as a bag.

In yet a further exemplary embodiment, the fluid-absorbing articles described herein may be used in “cell-saver” or hemodynamic systems. “Cell-saver” systems provide hemoglobin or other nutrients into blood and then filter the blood to improve the quality of the blood. Hemodynamic systems relate to blood flow through a patient. In either type of system, waste or unusable blood is collected. The collected blood may be immobilized using the fluid-absorbing articles described herein.

In any of the above-described application or any other suitable application for immobilizing a fluid, the fluid-absorbing articles described herein may be inputted into a container prior to or after introduction of a given fluid into the container. In most applications, it is desirable for the fluid-absorbing article to be added to the fluid in the container. As soon as the fluid-absorbing article come into contact with the fluid, the water-soluble pouch of the fluid-absorbing article begins to solubilize. Due to the density of the fluid-absorbing article, the fluid-absorbing article floats on or near an upper surface of the fluid (depending on the size and configuration of the container and the fluid-absorbing article). Typically, within 1 to 5 seconds, the water-soluble pouch of the fluid-absorbing article breaks open to allow contact between the superabsorbent material within the pouch and the fluid within the container. As superabsorbent material distributes throughout the container, fluid within the container is immobilized.

The fluid-absorbing articles of the present invention may have any dimensions and shape depending on the intended use of the product. For example, when used to immobilize fluids in a canister as described above, the fluid-absorbing article may have a tubular or cylindrical shape. It should be noted that the fluid-absorbing articles of the present invention may have any other geometric shape including, but not limited to, a rectangular shape, a cubical shape, a spherical shape, etc.

The fluid-absorbing articles may be sized depending on the amount of fluid to be immobilized. Typically, the fluid-absorbing article contains about 1 part particulate superabsorbent material for every 25 to 35 parts of fluid to be immobilized. For example, when used to immobilize 1200 cc of fluid in a 1500 cc canister, the fluid-absorbing article may contain about from about 34 to about 48 grams of particulate superabsorbent material (i.e., 34 to 48 g of particulate superabsorbent material for about 1200 g of fluid). When used to immobilize 2800 cc of fluid in a 3000 cc canister, the fluid-absorbing article may contain from about 80 to about 112 grams of particulate superabsorbent material (i.e., 80 to 112 g of particulate superabsorbent material for about 2800 g of fluid).

The fluid-absorbing articles of the present invention typically contain up to about 400 g of particulate superabsorbent material, but may contain any amount of particulate superabsorbent material up to about 400 g or greater than 400 g. In many applications, the fluid-absorbing articles of the present invention contain from about 30 to about 200 g of particulate superabsorbent material, and other components as described above. The resulting fluid-absorbing article may have outer dimensions to provide a water-soluble pouch having a pouch volume of up to about 515 cc, although most applications require smaller pouch volumes. For example, in one exemplary embodiment, a fluid-absorbing article for use with a fluid-containing canister as described above may have a tubular shape with a diameter of 2.54 cm and a length of about 7.6 cm, resulting in a pouch volume of about 38.5 cc.

The fluid-absorbing articles described herein may have any desired shape and configuration. Suitable shapes include, but are not limited to, a tubular shape, a spherical shape, a barbell shape, a star shape, a brick shape, or any other shape. The cross-sectional configuration of the fluid-absorbing articles may include any cross-sectional shape including, but not limited to, a circular shape, a triangular shape, a square shape, a rectangular shape, or any other shape. In one exemplary embodiment, the fluid-absorbing articles have an overall length greater than a cross-sectional dimension so that the fluid-absorbing article can have a desired pouch volume, and still have a cross-sectional area that allows the fluid-absorbing article to be inserted into an opening of a container. As described above, fluid-absorbing articles having a tubular shape are suitable for a number of fluid immobilizing applications.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

### Preparation of a Fluid-Absorbing Article

A fluid-absorbing article was prepared as follows. A sheet of MONOSOL® PVA film material commercially available from Monosol USA (Portage, Ind.) having an average film thickness of 38 μm (1.5 mil) was cut to form a first pouch-forming sheet having dimensions: length—15.2 cm (6.0 in) and width—5.0 cm (2.0 in). The first pouch-forming sheet was positioned in a cavity within a metal substrate. About 225 grams of superabsorbent particles (sodium polyacrylate having an average particle size of less than about 300 μm) commercially available from Emerging Technologies, Inc. (Greensboro, N.C.) under the trade designation NORSOCRYL XFS was placed into the lined cavity to completely fill the cavity.

A water spray was used to coat a fine mist of water onto outer edges of the first pouch-forming sheet extending around the edges of the cavity. A second pouch-forming sheet having dimensions: length—15.2 cm (6.0 in) and width—3.0 cm (1.2 in) was placed over the cavity containing the superabsorbent particles, and aligned with outer edges of the first pouch-forming sheet. Heat and pressure was applied to the edges to bond edges of the first pouch-forming sheet to edges of the second pouch-forming sheet. The resulting fluid-absorbing article was removed from the cavity.

The resulting fluid-absorbing article had an amount of air within the pouch volume of less than about 3 vol % based on a total pouch volume.

### EXAMPLE 2

### Preparation of a Fluid-Absorbing Article

A fluid-absorbing article was prepared using the procedure of Example 1 except a 9:1 (parts by weight) mixture of NORSOCRYL XFS superabsorbent particles and 2,2-dibromo-3-nitrilopropionamide (DBNPA) commercially available from Dow Chemical (Midland, Mich.) was placed into the lined cavity, and sealed to form a fluid-absorbing article.

The resulting fluid-absorbing article had an amount of air within the pouch volume of less than about 5 vol % based on a total pouch volume.

### EXAMPLE 3

### Preparation of a Fluid-Absorbing Article

A fluid-absorbing article was prepared using the procedure of Example 1 except a vacuum line was connected to the resulting fluid-absorbing article. The vacuum line pierced the PVA pouch, and removed any air present in the pouch. As the vacuum line exited the PVA pouch, the opening was resealed using heat and pressure.

The resulting fluid-absorbing article had an amount of air within the pouch volume of less than about 1 vol % based on a total pouch volume.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A fluid-absorbing article comprising:
particulate superabsorbent material; and
a sealed water-soluble pouch encapsulating the particulate superabsorbent material, wherein the sealed water-soluble pouch comprises a water-soluble film that is formed around the particulate superabsorbent material to minimize the amount of air within the sealed water-soluble pouch.

2. The fluid-absorbing article of claim 1, wherein the particulate superabsorbent material has an average particle size ranging from about 150 μm to about 800 μm.

3. The fluid-absorbing article of claim 1, wherein the fluid-absorbing article contains less than about 1.0 wt % water based on a total weight of the fluid-absorbing article.

4. The fluid-absorbing article of claim 1, further comprising at least one antimicrobial component within the sealed water-soluble pouch.

5. The fluid-absorbing article of claim 1, wherein the fluid-absorbing article consists essentially of particulate superabsorbent material, and the sealed water-soluble pouch consists essentially of water-soluble film.

6. The fluid-absorbing article of claim 1, wherein the water-soluble film has an average film thickness of up to about 51 microns.

7. The fluid-absorbing article of claim 1, wherein the sealed water-soluble pouch comprises a water-soluble polyvinyl alcohol film.

8. The fluid-absorbing article of claim 1, wherein the sealed water-soluble pouch consists essentially of polyvinyl alcohol.

9. The fluid-absorbing article of claim 1 in combination with a suction canister for use in an operating room environment.

10. A fluid-absorbing article consisting essentially of:
particulate superabsorbent material; and
a water-soluble pouch encapsulating the particulate superabsorbent material, wherein the water-soluble pouch consists essentially of water-soluble film.

11. The fluid-absorbing article of claim 10, wherein the pouch contains less than about 1.0 wt % water based on a total weight of the fluid-absorbing article.

12. The fluid-absorbing article of claim 10, wherein the pouch contains less than about 3.0 vol % air based on a total volume encapsulated by the water-soluble pouch.

13. The fluid-absorbing article of claim 10, wherein the water-soluble film consists essentially of polyvinyl alcohol.

14. The fluid-absorbing article of claim 10, wherein the water-soluble film has an average film thickness of up to about 51 microns.

15. The fluid-absorbing article of claim 10 in combination with a suction canister for use in an operating room environment.

16. A fluid-absorbing article consisting essentially of:
a mixture of (a) particulate superabsorbent material and (b) at least one of (i) a particulate antimicrobial component, (ii) an antimicrobial component coated onto the particulate superabsorbent material, or (iii) an antimicrobial component coated onto a particulate support consisting of carbon black; and
a water-soluble pouch encapsulating the mixture, wherein the water-soluble pouch consists essentially of water-soluble film material.

17. The article of claim 16, wherein the at least one antimicrobial dialdehyde consists essentially of glutaraldehyde (GLUT) alone or in combination with orthophthaldehyde (OPA).

18. The article of claim 16, wherein the pouch contains from about 80 wt % to about 99 wt % of particulate superabsorbent material, from about 20 wt % to about 1.0 wt % of at least one antimicrobial component, and less than about 1.0 wt % water based on a total weight of the superabsorbent material and the at least one antimicrobial component.

19. The article of claim 16, wherein the water-soluble film is shrink-wrapped around the superabsorbent material and the at least one antimicrobial component such that the water-soluble pouch substantially conforms to a mass containing the superabsorbent material and the at least one antimicrobial component.

20. The article of claim 16, wherein the water-soluble pouch contains less than about 3.0 vol % air based on a total volume encapsulated by the water-soluble pouch.

21. A method of immobilizing a fluid, said method comprising the steps of:
collecting one or more fluids in a container; and
introducing the fluid-absorbing article of claim 1 into the container.

22. The method of claim 21, wherein the container comprises a canister, a bag, or any other container capable of at least temporarily containing an aqueous fluid.

23. The method of claim 21, wherein the container comprises a suction canister, and the one or more fluids are generated during a procedure in an operating room.

24. The method of claim 21, wherein the container comprises a bag, and the one or more fluids comprise urine, blood, emesis or any other body fluid.

25. The method of claim 21, wherein the sealed water-soluble pouch comprises water-soluble polyvinyl alcohol film.

26. The method of claim 21, wherein the sealed water-soluble pouch consists essentially of water-soluble polyvinyl alcohol film.
